(19) United States
(12) Patent Application Publication
(10) Pub. No.: US 2010/0221221 A1
(43) Pub. Date: Sep. 2, 2010

(54) N-PHENYL-2-PYRIMIDINEAMINE DERIVATIVES

(75) Inventors: Scott L. Harbeson, Cambridge, MA (US); Julie F. Liu, Lexington, MA (US); Roger Tung, Lexington, MA (US)

Correspondence Address: FISH & RICHARDSON P.C., PO BOX 1022, MINNEAPOLIS, MN, 55440-1022, US

(73) Assignee: CONCERT PHARMACEUTICALS INC., Lexington, MA (US)

(21) Appl. No.: 12/539,091

(22) Filed: Aug. 11, 2009

Related U.S. Application Data
(60) Provisional application No. 61/088,068, filed on Aug. 12, 2008.

(51) Int. Cl. A61K 38/21; C07D 401/14; A61K 31/506; A61P 35/02; A61P 35/00; A61P 33/06; A61P 35/04; A61P 27/14; A61P 19/02; A61P 13/12; A61P 15/00; A61P 17/06; A61P 3/10; A61P 9/10; A61P 17/10; A61P 31/12; A61P 13/06; A61P 31/18; A61K 39/395; C12N 5/02; A61K 31/55; A61K 38/50; A61K 31/661; A61K 31/7068; A61K 35/12; A61K 33/24

(52) U.S. Cl. 424/85.7; 544/295; 514/252.18; 424/133.1; 435/375; 514/214.01; 424/94.6; 514/110; 514/49; 424/93.71; 424/649; 424/158.1

(57) ABSTRACT

This disclosure relates to novel N-phenyl-2-pyrimidineamines and pharmaceutically acceptable salts thereof. This disclosure also provides compositions comprising a compound of this disclosure and the use of such compositions in methods of treating diseases and conditions that are beneficially treated by administering protein-tyrosine kinase inhibitors.

### RELATED APPLICATIONS

This application claims the benefit of U.S. Provisional Patent Application No. 61/088,068, filed Aug. 12, 2008. The above application is incorporated herein by reference in its entirety.

### TECHNICAL FIELD

This disclosure relates to novel N-phenyl-2-pyrimidineamines and pharmaceutically acceptable salts thereof. This disclosure also provides compositions comprising a compound of this disclosure and the use of such compositions in methods of treating diseases and conditions that are beneficially treated by administering protein-tyrosine kinase inhibitors.

### BACKGROUND

Imatinib, also known as 4-[(4-Methyl-1-piperazinyl)methyl]-N-[4-methyl-3-[[4-(3-pyridinyl)-2-pyrimidinyl]amino]-phenyl]benzamide, is a protein-tyrosine kinase inhibitor which inhibits the bcr-abl tyrosine kinase, the receptor tyrosine kinases for platelet-derived growth factor (PDGF) and stem cell factor (SCF), and c-kit, in addition to inhibiting PDGF- and SCF-mediated cellular events.

Imatinib is currently approved for chronic myeloid leukemia, gastrointestinal stromal cancer (GIST), fibrosarcoma, acute lymphocytic leukemia, hypereosinophilic syndrome, myeloproliferative diseases, and systemic mastocytosis, and in clinical trials for astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak glomerulosclerosis (FSGS), radiation-induced fibrosis such as osteoradionecrosis, multiple sclerosis, and uterine leiomyomas (fibroids).

The main circulating metabolite of imatinib, the N-demethylated piperazine derivative, has similar activity to the parent drug and is formed predominantly by CYP3A4. The half-lives of imatinib and this metabolite are 18 hours and 40 hours respectively, with the metabolite having a plasma AUC approximately 15% that of the parent. Other cytochrome P450 enzymes playing a minor role in metabolism of imatinib include CYP1A2, CYP2D6, CYP2C9, and CYP2C19. (See FDA label for imatinib at http://www.fda.gov/cder/foi/label/2006/021588s0091bl.pdf).

Adverse events associated with the use of imatinib include, but are not limited to, edema, nausea, vomiting, muscle cramps, musculoskeletal pain, diarrhea, and rash. (See FDA label: http://www.fda.gov/cder/foi/label/2006/021588s0091bl.pdf).

Despite the beneficial activities of imatinib, there is a continuing need for new compounds to treat the aforementioned diseases and conditions.

### SUMMARY

Provided herein is a compound of Formula I:

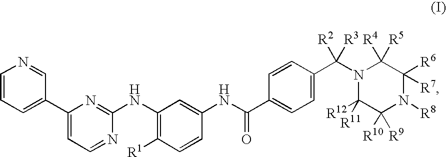

or a pharmaceutically acceptable salt thereof, wherein:

R¹ is selected from CH₃, CH₂D, CHD₂, and CD₃;

each of R², R³, R⁴, R⁵, R⁶, R⁷, R⁹, R¹⁰, R¹¹ and R¹² is independently selected from hydrogen and deuterium;

R⁸ is selected from hydrogen, CH₃, CH₂D, CHD₂, and CD₃; and

at least one R comprises a deuterium atom,

wherein:

when R⁸ is CD₃, then at least one additional R comprises a deuterium atom;

when R² and R³ are simultaneously hydrogen, R⁴, R⁵, R⁶, R⁷, R⁹, R¹⁰, R¹¹ and R¹² are

when R² and R³ are simultaneously hydrogen, R⁴, R⁵, simultaneously deuterium and R⁸ is hydrogen, then R¹ comprises a deuterium atom; and

when R² and R³ are simultaneously hydrogen, R⁴, R⁵, R⁶, R⁷, R⁹, R¹⁰, R¹¹ and R¹² are simultaneously deuterium, R¹ is CH₃, and R⁸ is CH₃, then all carbon atoms are present at their natural isotopic abundance.

In some embodiments of Formula I, R⁸ is selected from hydrogen, CH₃, and CD₃. In some embodiments, R⁸ is hydrogen.

Also provided is a compound of Formula II:

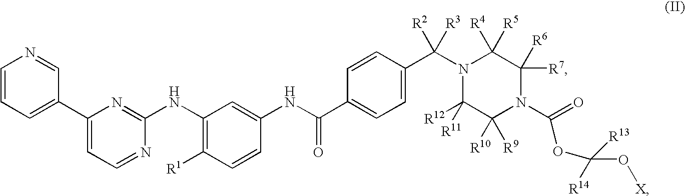

or a pharmaceutically acceptable salt thereof, wherein:

R¹ is selected from CH₃, CH₂D, CHD₂, and CD₃;

each of R², R³, R⁴, R⁵, R⁶, R⁷, R⁹, R¹⁰, R¹¹ and R¹² is independently selected from hydrogen and deuterium;

each of R¹³ and R¹⁴ is independently selected from hydrogen, deuterium, C₁-C₆-alkyl, C₂-C₆ alkenyl, C₂-C₆ alkynyl, C₃-C₆ cycloalkyl, C₅-C₆ cycloalkenyl, and C₄-C₈ (cycloalkyl)alkyl; or

R¹³ and R¹⁴ are taken together with the carbon atom to which they are bound to form a 3-to 7-membered carbocyclic ring; and

X is selected from —PO₃H₂ (including a pharmaceutically acceptable salt of —PO₃H₂) and -A-R¹⁵, wherein A is an α-amino acid residue and R¹⁵ is selected from hydrogen, —CH₃, —C(O)CH₃, and an α-amino acid.

In some embodiments of Formula II, each of R¹³ and R¹⁴ is independently selected from hydrogen and —CH₃.

In some embodiments of Formula II, X is selected from a pharmaceutically acceptable salt of —PO₃H₂ or -A-R¹⁵, wherein A is a naturally occurring α-amino acid and R¹⁵ is hydrogen.

In some embodiments of Formula II, X is a pharmaceutically acceptable salt of —PO₃H₂.

In some embodiments of Formula I or Formula II, each R group attached to a common carbon atom is the same.

In some embodiments of Formula I or Formula II, R⁴, R⁵, R⁶, R⁷⁵, R⁹, R¹⁰, R¹¹ and R¹² are the same.

In some embodiments of Formula I or Formula II, R¹ is selected from CH₃ and CD₃.

In some embodiments of Formula I, R² and R³ are the same; each of R⁴, R⁵, R⁶, R⁷, R⁹, R¹⁰, R¹¹ and R¹² are the same, and the compound is selected from any one of the compounds set forth in the table:

| Compound | R¹ | R²=R³ | R⁴=R⁵=R⁶=R⁷=R⁹=R¹⁰=R¹¹=R¹² | R⁸ |
|---|---|---|---|---|
| 100 | CD₃ | D | D | CD₃ |
| 101 | CD₃ | D | D | CH₃ |
| 102 | CD₃ | D | D | H |
| 103 | CD₃ | H | D | CD₃ |
| 104 | CD₃ | H | D | CH₃ |
| 105 | CD₃ | H | D | H |
| 106 | CD₃ | D | H | CD₃ |
| 107 | CD₃ | D | H | CH₃ |
| 108 | CD₃ | D | H | H |
| 109 | CH₃ | D | D | CD₃ |
| 110 | CH₃ | D | D | CH₃ |
| 111 | CH₃ | D | D | H |
| 112 | CH₃ | H | D | CD₃ |
| 113 | CH₃ | H | D | CH₃ |
| 114 | CH₃ | H | D | H |

or a pharmaceutically acceptable salt of any of the foregoing.

In one embodiment of Formula I, the compound of this invention is

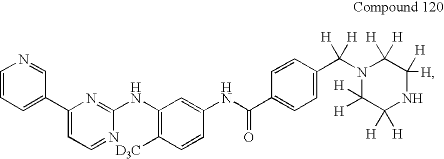

or a pharmaceutically acceptable salt thereof.

In some embodiments of Formula II, R² and R³ are the same; each of R⁴, R⁵, R⁶, R⁷, R⁹, R¹⁰, R¹¹ and R¹² is the same; and R¹³ and R¹⁴ are hydrogen; where the compound is selected from any one of the compounds set forth in the table:

| | | | R⁴=R⁵=R⁶= | |
|---|---|---|---|---|
| Compound | R¹ | R²=R³ | R⁷=R⁹=R¹⁰=R¹¹=R¹² | X |
| 115 | CD₃ | D | H | PO₃H₂ |
| 116 | CD₃ | H | H | PO₃H₂ |
| 117 | CD₃ | D | D | PO₃H₂ |
| 118 | CH₃ | D | H | PO₃H₂ |
| 119 | CH₃ | H | H | PO₃H₂ |

or a pharmaceutically acceptable salt of any of the foregoing.

In some embodiments of Formula I or II, any atom not designated as deuterium is present at its natural isotopic abundance.

Also provided is a pyrogen-free composition comprising:

a) a compound of Formula I:

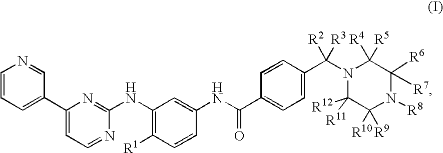

or a pharmaceutically acceptable salt thereof, wherein:

R¹ is selected from CH₃, CH₂D, CHD₂, and CD₃;

each of R², R³, R⁴, R⁵, R⁶, R⁷, R⁹, R¹⁰, R¹¹ and R¹² are independently selected from hydrogen and deuterium;

R⁸ is selected from hydrogen, CH₃, CH₂D, CHD₂, and CD₃; and

at least one R comprises a deuterium atom; and

b) an acceptable carrier.

In one embodiment of the pyrogen-free composition, when R² and R³ are simultaneously hydrogen, R⁴, R⁵, R⁶, R⁷, R⁹, R¹⁰, R¹¹ and R¹² are simultaneously deuterium and R is hydrogen, then R¹ comprises a deuterium atom; and when R² and R³ are simultaneously hydrogen, R⁴, R⁵, R⁶, R⁷, R⁹, R¹⁰, R¹¹ and R¹² are simultaneously deuterium, R¹ is CH₃, and R⁸ is CH₃, then all carbon atoms are present at their natural isotopic abundance.

Further provided is a pyrogen-free composition comprising:

a) a compound of Formula II:

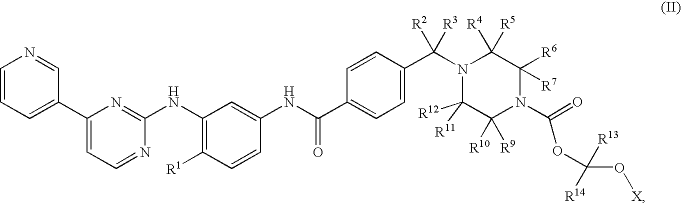

or a pharmaceutically acceptable salt thereof, wherein:

R¹ is selected from CH₃, CH₂D, CHD₂, and CD₃;

each of R², R³, R⁴, R⁵, R⁶, R⁷, R⁹, R¹⁰, R¹¹ and R¹² is independently selected from hydrogen and deuterium;

each of R¹³ and R¹⁴ is independently selected from hydrogen, deuterium, C₁-C₆-alkyl, C₂-C₆ alkenyl, C₂-C₆ alkynyl, C₃-C₆ cycloalkyl, C₅-C₆ cycloalkenyl, and C₄-C₈ (cycloalkyl)alkyl; or

R¹³ and R¹⁴ are taken together with the carbon atom to which they are bound to form an optionally substituted 3- to 7-membered carbocyclic ring; and

X is selected from —PO₃H₂ (including a pharmaceutically acceptable salt of —PO₃H₂) and -A-R¹⁵, wherein A is an α-amino acid residue and R¹⁵ is selected from hydrogen, —CH₃, —C(O)CH₃, and an α-amino acid; and

b) an acceptable carrier.

In some embodiments, a composition is formulated for pharmaceutical administration, and the carrier is a pharmaceutically acceptable carrier.

In some embodiments, a composition comprises a second therapeutic agent, wherein the second therapeutic agent is useful to treat a patient suffering from or susceptible to chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

In some embodiments, the second therapeutic agent is selected from hydroxyurea, ranibizumab, homoharringtonine, asparaginase, cyclophosphamide, cytarabine, daunorubicin hydrochloride, etoposide, filgrastim, idarubicin, mercaptopurine, methotrexate, methylprednisolone, mitoxantrone hydrochloride, prednisone, vincristine, TALL-104 cells, cladribine, temsirolimus, alemtuzumab, IFN-alpha, busulfan, fludarabine, clofarabine, xeloda, pioglitazone, etoricoxib, dexamethason, treosulfan, docetaxel, sunitinib, vinorelbine, cisplatin, pemetrexed, temozolomide, vatalanib, everolimus, taxotere, gemcitabine, and capecitabine pentoxyfylline, pirfenidone, and antibodies against TGF-beta, CTGF, and alpha-v-beta-6.

Further provided is a method of inhibiting protein-tyrosine kinase activity in a cell, comprising contacting the cell with a compound of Formula I or Formula II.

A method of treating a patient is also provided. The patient may be suffering from, or susceptible to, a disease selected from skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids). The method includes the step of administering to the patient in need thereof a pharmaceutical composition of Formula I or Formula II.

In some embodiments of the treatment method, the patient is suffering from or susceptible to a disease or condition selected from chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

In some embodiments of the treatment method, the patient is suffering from or susceptible to a disease or condition selected from chronic myeloid leukemia, gastrointestinal stromal cancer (GIST), fibrosarcoma, acute lymphocytic leukemia, hypereosinophilic syndrome, myeloproliferative diseases, and systemic mastocytosis.

In some embodiments of a treatment method, an additional step of administering to the patient in need thereof a second therapeutic agent is included, wherein the second therapeutic agent is useful to treat a condition selected from: chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

In some embodiments of the method, for example:

a) the patient is suffering from of susceptible to leukemia; and the second therapeutic agent is selected from homoharringtonine, asparaginase, cyclophosphamide, cytarabine, daunorubicin hydrochloride, etoposide, filgrastim, idarubicin, mercaptopurine, methotrexate, methylprednisolone, mitoxantrone hydrochloride, prednisone, vincristine, TALL-104 cells, cladribine, temsirolimus, alemtuzumab, IFNalpha, busulfan, fludarabine, and clofarabine;

b) the patient is suffering from of susceptible to glioblastoma multiforme; and the second therapeutic agent is hydroxyurea;

c) the patient is suffering from of susceptible to choroidal neovascularization; and the second therapeutic agent is ranibizumab;

d) the patient is suffering from of susceptible to colorectal cancer; and the second therapeutic agent is xeloda;

e) the patient is suffering from of susceptible to prostate cancer; and the second therapeutic agent is selected from pioglitazone, etoricoxib, dexamethason, treosulfan, and docetaxel;

f) the patient is suffering from of susceptible to gastrointestinal stromal tumor; and the second therapeutic agent is sunitinib;

g) the patient is suffering from of susceptible to breast cancer; and the second therapeutic agent is selected from vinorelbine and docetaxel;

h) the patient is suffering from of susceptible to mesothelioma; and the second therapeutic agent is selected from cisplatin and pemetrexed;

i) the patient is suffering from of susceptible to brain and central nervous system tumors or glioma; and the second therapeutic agent is selected from temozolomide, vatalanib, and hydroxyurea;

j) the patient is suffering from of susceptible to renal cancer; and the second therapeutic agent is everolimus;

k) the patient is suffering from of susceptible to head and neck cancer; and the second therapeutic agent is docetaxel;

l) the patient is suffering from of susceptible to lung cancer; and the second therapeutic agent is taxotere;

m) the patient is suffering from of susceptible to solid tumors; and the second therapeutic agent is selected from gemcitabine and capecitabine.

### DETAILED DESCRIPTION OF THE DISCLOSURE

The terms “ameliorate” and “treat” are used interchangeably and include therapeutic and/or prophylactic treatment. Both terms mean decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of imatinib will inherently contain small amounts of deuterated and/or ¹³C-containing isotopologues. The concentration of naturally abundant stable hydrogen and carbon isotopes, notwithstanding this variation, is small and immaterial as compared to the degree of stable isotopic substitution of compounds of this disclosure. See, for instance, Wada E et al., Seikagaku 1994, 66:15; Gannes L Z et al., Comp Biochem Physiol Mol Integr Physiol 1998, 119:725. In a compound of this disclosure, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

Not withstanding the above, unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

The term “isotopologue” refers to a species that has the same chemical structure and formula as a specific compound of this disclosure, with the exception of the isotopic composition at one or more positions, e.g., H vs. D, and/or the level of isotopic enrichment at one or more positions.

The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of deuterated reagents used to make the compound and the efficiency of incorporation of deuterium in the various synthesis steps used to prepare the compound. However, as set forth above the relative amount of such isotopologues will be less than 49.9% of the compound.

The term “compound,” as used herein, is also intended to include any salts thereof.

A salt of a compound of this disclosure is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The disclosed compounds may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms.

When the stereochemistry of the disclosed compounds is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% optically pure. Percent optical purity by weight is the ratio of the weight of the enantiomer over the weight of the enantiomer plus the weight of its optical isomer.

When a disclosed compound is named or depicted by structure without indicating the stereochemistry, and has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer (“scalemic mixtures”).

When a disclosed compound is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert” and “t-” each refer to tertiary. “US” refers to the United States of America. “FDA” refers to Food and Drug Administration. “NDA” refers to New Drug Application.

The term “methylene” refers to —CH₂—. The term “methenylene” refers to ═CH— or —CH═.

The term “α-amino acid residue” refers to a group of the general formula —C(O)—CHR—NH— and N-alkylated derivatives thereof (—C(O)—CHR—N(alkyl)- and includes naturally occurring and synthetic amino acids in either a (D)-, (L)- or racemic (D,L) configuration. It is understood that when the variable A in Formula II, below, is an α-amino acid residue, it is linked to the rest of the molecule through the carbonyl carbon directly bonded to the α-carbon of the amino acid. In accordance with the structure of Formula II, such a linkage results in the formation of an ester.

The term “α-amino acid” refers to a terminal group of the general formula —C(O)—CHR—NH₂ or —C(O)—CHR—NH(alkyl) and includes naturally occurring and synthetic amino acids in either a (D)-, (L)- or racemic (D,L) configuration.

The term “carbocyclic ring” refers to an unsaturated, partially saturated, aromatic, or fully saturated, monocyclic ring wherein all ring atoms are carbon. Examples include cycloalkyl, cycloalkenyl, and phenyl rings. Carbocyclic rings may be optionally substituted with one or more substituents independently selected from —OH, O—(C₁-C₄ alkyl), —NH₂, —NH(C₁-C₄ alkyl) and N(C₁-C₄ alkyl)₂.

Throughout this specification, a variable may be referred to generally (e.g.,“each R”) or may be referred to specifically (e.g., R¹, R², R³, etc.). Unless otherwise indicated, when a variable is referred to generally, it is meant to include all specific embodiments of that particular variable.

### Therapeutic Compounds

The present disclosure provides a compound of Formula I:

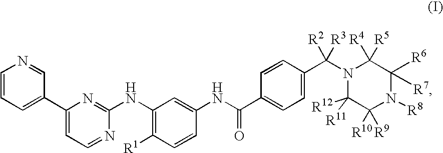

or a pharmaceutically acceptable salt thereof, wherein:

R¹ is selected from CH₃, CH₂D, CHD₂, and CD₃;

each of R², R³, R⁴, R⁵, R⁶, R⁷, R⁹, R¹⁰, R¹¹ and R¹² is independently selected from hydrogen and deuterium;

R⁸ is selected from hydrogen, CH₃, CH₂D, CHD₂, and CD₃; and

at least one R comprises a deuterium atom,

wherein:

when R⁸ is CD₃, then at least one additional R comprises a deuterium atom;

when R² and R³ are simultaneously hydrogen, R⁴, R⁵, R⁶, R⁷, R⁹, R¹⁰, R¹¹ and R¹² are simultaneously deuterium and R⁸ is hydrogen, then R¹ comprises a deuterium atom; and

when R² and R³ are simultaneously hydrogen, R⁴, R⁵, R⁶, R⁷, R⁹, R¹⁰, R¹¹ and R¹² are simultaneously deuterium, R¹ is CH₃, and R⁸ is CH₃, then all carbon atoms are present at their natural isotopic abundance.

Additional embodiments of compounds of Formula I include those wherein:

a) each R group attached to a common carbon atom (e.g., R² and R³; R⁴ and R⁵; R⁶ and R⁷_{; R}⁹ and R¹⁰; R¹¹ and R¹²) is the same;

b) R⁴, R⁵, R⁶, R⁷, R⁹, R¹⁰, R¹¹ and R¹² are the same;

c) R⁸ is selected from hydrogen, CH₃, and CD₃; and

d) R¹ is selected from CH₃, and CD₃.

In one embodiment, R⁸ is hydrogen.

Another embodiment provides compounds of Formula I having at least two features selected from (a), (b), (c) and (d) listed above.

Examples of specific compounds of Formula I, wherein R² and R³ are the same; and each of R⁴, R⁵, R⁶, R⁷, R⁹, R¹⁰, R¹¹ and R¹² is the same are shown in Table 1 below.

Examples of Specific Compounds of Formula I

| Compound | R¹ | R²=R³ | R⁴=R⁵=R⁶=R⁷=R⁹=R¹⁰=R¹¹=R¹² | R⁸ |
|---|---|---|---|---|
| 100 | CD₃ | D | D | CD₃ |
| 101 | CD₃ | D | D | CH₃ |
| 102 | CD₃ | D | D | H |
| 103 | CD₃ | H | D | CD₃ |
| 104 | CD₃ | H | D | CH₃ |
| 105 | CD₃ | H | D | H |
| 106 | CD₃ | D | H | CD₃ |
| 107 | CD₃ | D | H | CH₃ |
| 108 | CD₃ | D | H | H |
| 109 | CH₃ | D | D | CD₃ |
| 110 | CH₃ | D | D | CH₃ |
| 111 | CH₃ | D | D | H |
| 112 | CH₃ | H | D | CD₃ |
| 113 | CH₃ | H | D | CH₃ |
| 114 | CH₃ | H | D | H |

or a pharmaceutically acceptable salt thereof.

In another embodiment of Formula I, the compound of this invention is

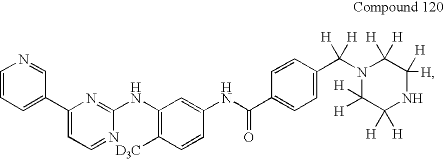

or a pharmaceutically acceptable salt thereof.

In another embodiment, the disclosure provides a compound of Formula II:

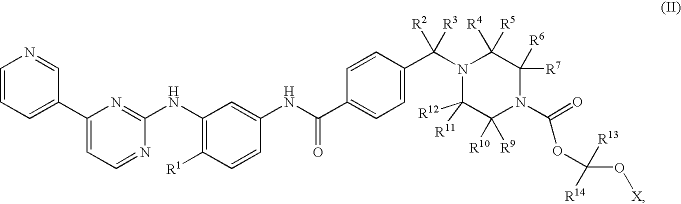

or a pharmaceutically acceptable salt thereof, wherein:

R¹ is selected from CH₃, CH₂D, CHD₂, and CD₃;

each of R², R³, R⁴, R⁵, R⁶, R⁷, R⁹, R¹⁰, R¹¹ and R¹² is independently selected from hydrogen and deuterium;

each of R¹³ and R¹⁴ is independently selected from hydrogen, deuterium, C₁-C₆-alkyl, C₂-C₆ alkenyl, C₂-C₆ alkynyl, C₃-C₆ cycloalkyl, C₅-C₆ cycloalkenyl, and C₄-C₈ (cycloalkyl)alkyl; or

R¹³ and R¹⁴ are taken together with the carbon atom to which they are bound to form a 3-to 7-membered carbocyclic ring; and

X is selected from —PO₃H₂ (including a pharmaceutically acceptable salt of —PO₃H₂) and -A-R¹⁵, wherein A is an α-amino acid residue and R¹⁵ is selected from hydrogen, —CH₃, —C(O)CH₃, and an α-amino acid.

In one embodiment, the disclosure provides a compound of Formula II wherein at least one R is a deuterium atom.

In certain embodiments, R¹ is selected from —CH₃ and —CD₃. In a more specific embodiment, R¹ is —CD₃.

In other embodiments, R² and R³ are the same.

In still other embodiments, R⁴, R⁵, R⁶, R⁷, R⁹, R¹⁰, R¹¹ and R¹² are the same. In a more specific embodiment R⁴, R⁵, R⁶, R⁷, R⁹, R¹⁰, R¹¹ and R¹² are simultaneously hydrogen.

In another embodiment, each of R¹³ and R¹⁴ is independently selected from hydrogen and —CH₃.

In yet other embodiments, X is selected from a pharmaceutically acceptable salt of —PO₃H₂ or -A-R¹⁵, wherein A is a naturally occurring α-amino acid residue and R¹⁵ is hydrogen. In a more specific embodiment, X is a pharmaceutically acceptable salt of —PO₃H₂. In one aspect of these embodiments of X, R¹ is —CD₃; and R² and R³ are the same. In another aspect of these embodiments of X, R¹ is —CD₃; R² and R³ are the same; and R⁴, R⁵, R⁶, R⁷, R⁹, R¹⁰, R¹¹ and R¹² are simultaneously hydrogen. In still another aspect of these embodiments of X, R¹ is —CD₃; R² and R³ are the same; R⁴, R⁵, R⁶, R⁷, R⁹, R¹⁰, R¹¹ and R¹² are simultaneously hydrogen; and each of R¹³ and R¹⁴ is independently selected from hydrogen and —CH₃.

Examples of specific compounds of Formula II, wherein R² and R³ are the same; each of R⁴, R⁵, R⁶, R⁷, R⁹, R¹⁰, R¹¹ and R¹² is the same; and R¹³ and R¹⁴ are hydrogen are shown in Table 2 below.

TABLE 2
Examples of Specific Compounds of Formula II

| | | | R⁴=R⁵=R⁶= | |
|---|---|---|---|---|
| Compound | R¹ | R²=R³ | R⁷=R⁹=R¹⁰=R¹¹=R¹² | X |
| 115 | CD₃ | D | H | PO₃H₂ |
| 116 | CD₃ | H | H | PO₃H₂ |
| 117 | CD₃ | D | D | PO₃H₂ |
| 118 | CH₃ | D | H | PO₃H₂ |
| 119 | CH₃ | H | H | PO₃H₂ |

In another set of embodiments, any atom not designated as deuterium in any of the embodiments of Formula I or II set forth above is present at its natural isotopic abundance.

In another set of embodiments, the compound of Formula I or Formula II is isolated or purified, e.g., the compound of Formula I or Formula II is present at a purity of at least 50.1% by weight (e.g., at least 52.5%, 54%, 55%, 60%, 65%, 70%, 75%, 80%, 85%, 90%, 95%, 97%, 98%, 98.5%, 99%, 99.5% or 99.9%) of the total amount of isotopologues of Formula I or Formula II present, respectively. Thus, in some embodiments, a composition comprising a compound of Formula I or Formula II can include a distribution of isotopologues of the compound, provided at least 50.1% of the isotopologues by weight are the recited compound.

In some embodiments, any position in the compound of Formula I or Formula II designated as having D has a minimum deuterium incorporation of at least 50.1% (e.g., at least 52.5%, at least 60%, at least 67.5%, at least 75%, at least 82.5%, at least 90%, at least 95%, at least 97%, at least 99%, or at least 99.5%) at the designated position(s) of the compound of Formula I or Formula II. Thus, in some embodiments, a composition comprising a compound of Formula I or Formula II can include a distribution of isotopologues of the compound, provided at least 50.1% of the isotopologues include a D at the designated position(s).

In some embodiments, a compound of Formula I or Formula II is “substantially free of” other isotopologues of the compound, e.g., less than 49.9%, less than 25%, less than 10%, less than 5%, less than 2%, less than 1%, or less than 0.5% of other isotopologues are present.

The synthesis of compounds of Formula I or Formula II can be readily achieved by synthetic chemists of ordinary skill with references to the schemes below, which illustrate how the present compounds may be prepared, as well as to the Examples below. Relevant procedures analogous to those of use for the preparation of compounds of Formula I, Formula II and intermediates thereof are disclosed, for instance in de Bree, F et al., Drugs Fut, 2001, 26(6):545; Zimmermann, J et al., Bioorg Med Chem Lett, 1997, 7(2):187; Szakacs, Z et al., J Med Chem, 2005, 48(1):249-255; PCT patent publications WO2004074502, WO 2004108699, WO2006061332, and WO2006071130; United States Patent publication US 2006149061; and U.S. Pat. No. 5,521,184.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

### Exemplary Synthesis

Compounds of Formula I may be prepared as shown in Scheme 1 below in a manner analogous to that described by Kompella, A. et al., in PCT International Application WO2004108699.

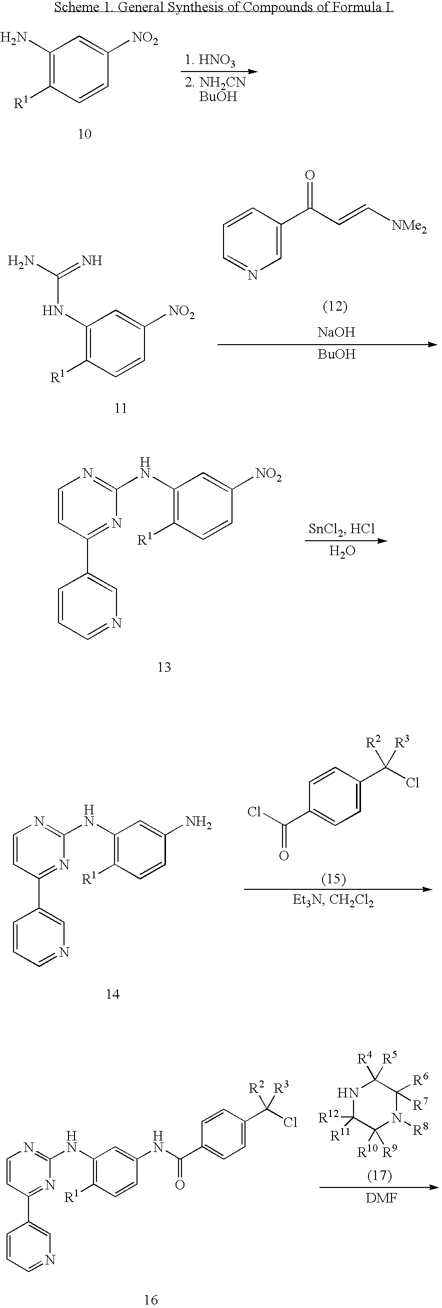

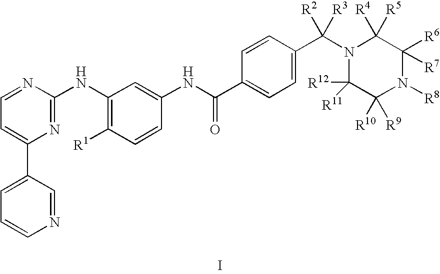

Scheme I above shows a general route for making compounds of Formula I. Treatment of nitroaniline 10 with nitric acid in butanol followed by condensation with cyanamide in butanol provides the guanidine salt 11. The pyrimidine intermediate 13 results from treatment of the guanidine salt 11 with commercially available compound 12 and NaOH in butanol. Reduction of pyrimidine intermediate 13 with tin chloride in a mixture of HCl and water provides the corresponding aniline 14. Acylation of aniline 14 with benzoyl chloride 15 can provide chloride 16. Chloride 16 may be treated with piperazine 17 in DMF to provide compounds of Formula I.

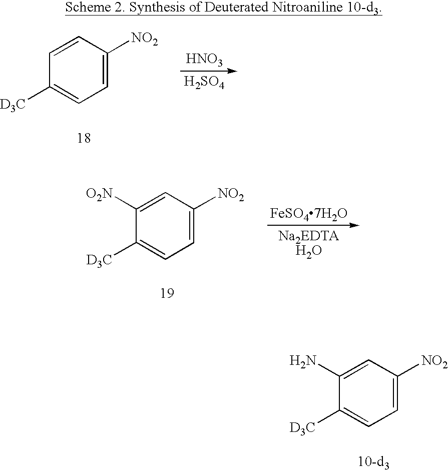

Scheme 2 above shows a route for making deuterated nitroaniline 10-d₃, which is useful for Scheme 1. Commercially available deuterated nitrobenzene 18 is converted to dinitrobenzene derivative 19 by treatment with a mixture of nitric acid and sulfuric acid using a procedure similar to that described in Synth Comm, 2001, 31(6): 823-826. Reduction of the ortho nitro group in compound 19 with hydrogen gas in the presence of ferrous sulfate and disodium EDTA in water under high pressure provides the deuterated nitroaniline 10-d₃ using a procedure similar to that described in J Org Chem, 2004, 69(14): 4835-4838.

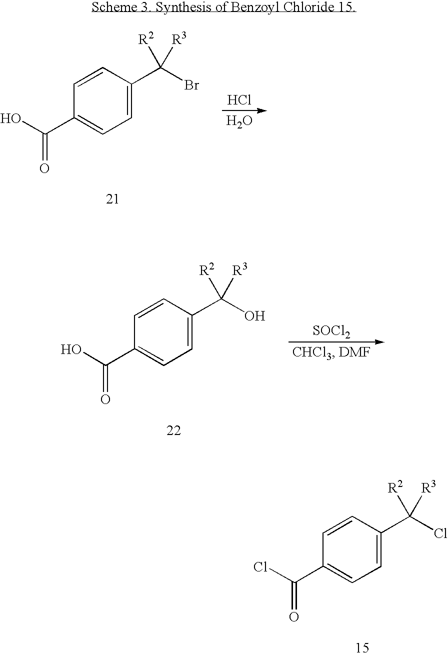

Scheme 3 above depicts a possible route to appropriately deuterated benzoyl chloride 15, of use in Scheme 1. Bromide 21 treated with aqueous HCl provides the corresponding alcohol 22. Benzoyl chloride 15 then may be prepared by treating alcohol 22 with thionyl chloride in a mixture of chloroform and DMF.

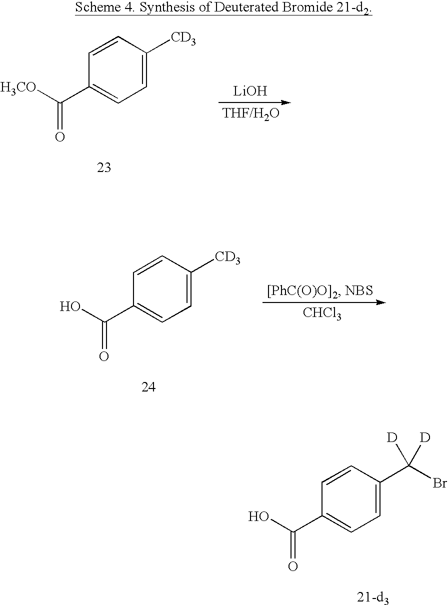

Scheme 4 above depicts a route for making deuterated bromide 21-d₂. Deuterated methyl benzoate 23 is prepared as described in Tet Lett, 2004, 45(31):5977-5981. Hydrolysis of the methyl ester of 23 with lithium hydroxide in a mixture of THF and water under reflux provides benzoic acid 24. Treatment of 24 with N-bromosuccinimide in the presence of benzoyl peroxide in chloroform provides the bromide 21-d₂.

Various deuterated N-methyl piperazines (17-d₈, 17-d₁₁, and 17-d₃) that are useful in Scheme 1 may be prepared as shown in Schemes 5a-5c below.

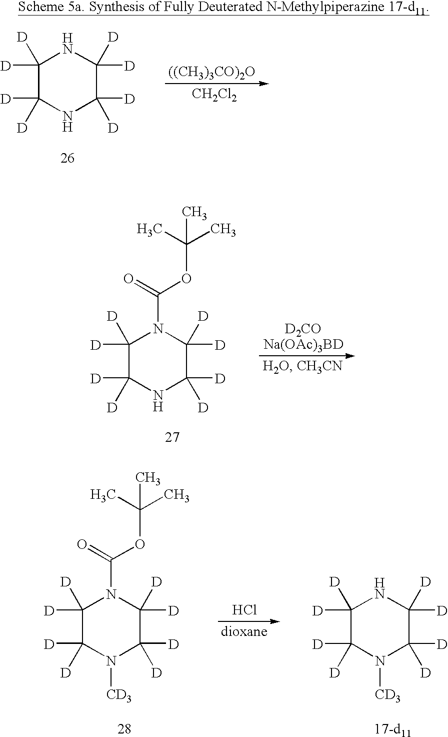

Scheme 5a above shows a route for preparing the fully deuterated N-methylpiperazine (17-d₁₁). Commercially available deuterated piperazine 26 may be treated with BOC anhydride (tert-butoxycarbonyl anhydride) in CH₂Cl₂ to prepare the BOC-protected piperazine 27 in a manner analogous to that described in J Med Chem, 2004, 47(17): 4300-4315. Reductive amination of 27 with deuterated formaldehyde and sodium triacetoxyborodeuteride in water and acetonitrile provides BOC-protected piperazine 28 using a procedure similar to that described in WO 2005044797. Removal of the BOC protecting group is accomplished by treating with HCl in dioxane to provide the deuterated piperazine 17-d₁₁.

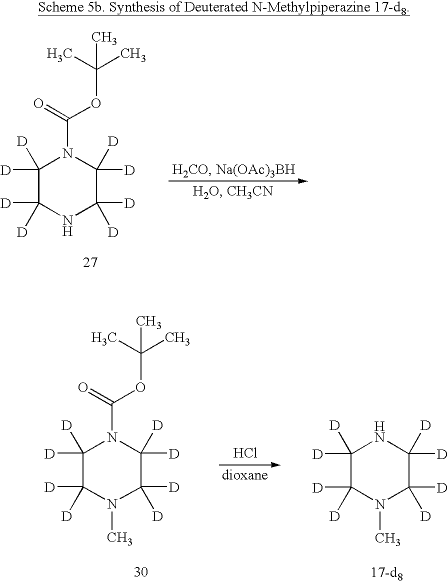

Scheme 5b above shows a route for making 17-d₈. Deuterated piperazine 17-d₈ may be prepared using a procedure similar that shown in Scheme 5a. BOC-piperazine 27 may be converted to N-methylated-Boc-piperazine 30 by treatment with formaldehyde and sodium triacetoxyborohydride. This is followed by removal of the BOC group by treatment with HCl in dioxane to produce 17-d₈.

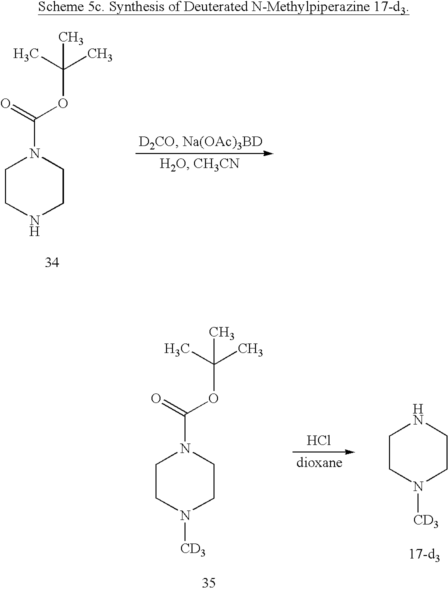

Scheme 5c above shows a route for making the deuterated piperazine 17-d₃. Reductive amination of commercially available amine 34 with deuterated formaldehyde and sodium triacetoxyborodeuteride in water and acetonitrile provides BOC-piperazine 35 using a procedure similar to that described in WO 2005044797. Removal of the BOC protecting group may be accomplished by treating with HCl in dioxane to provide 17-d₃.

When 27 is used as the deuterated piperazine to prepare analogs of compound I, the final step will be removal of the BOC group with HCl in dioxane.

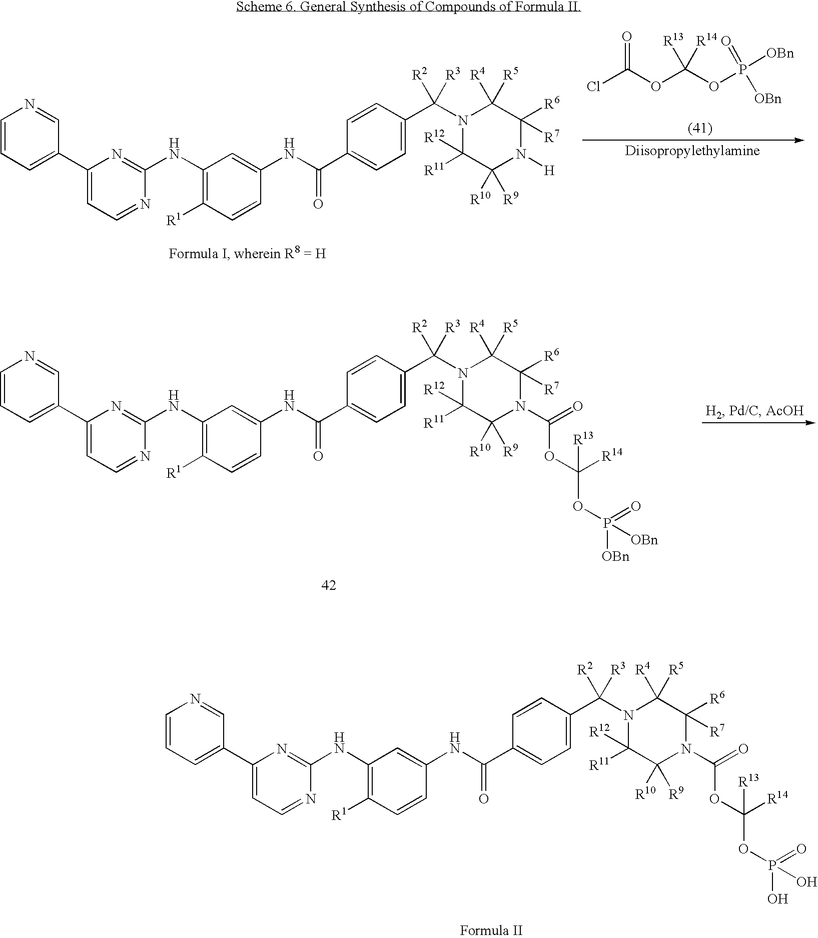

Scheme 6 depicts the preparation of compounds of Formula II. A compound of Formula I wherein R⁸ is hydrogen may be acylated with intermediate 41 such as known (bis(benzyloxy)phosphoryloxy)methyl carbonochloridate (41a, wherein R¹³ and R¹⁴═H; see PCT publication WO2007050732A1 and European Patent publication EP747385) optionally in the presence of a base such as diisopropylethylamine to provide carbamate 42. Removal of the benzyl protecting groups via hydrogenation over a palladium catalyst such as palladium on carbon or palladium hydroxide on carbon, optionally in the presence of an acid such as acetic acid, provides compounds of Formula II wherein X is —PO₃H₂. The disodium or calcium salt forms may be accessed via treatment with sodium hydroxide or with calcium hydroxide or calcium acetate or calcium chloride.

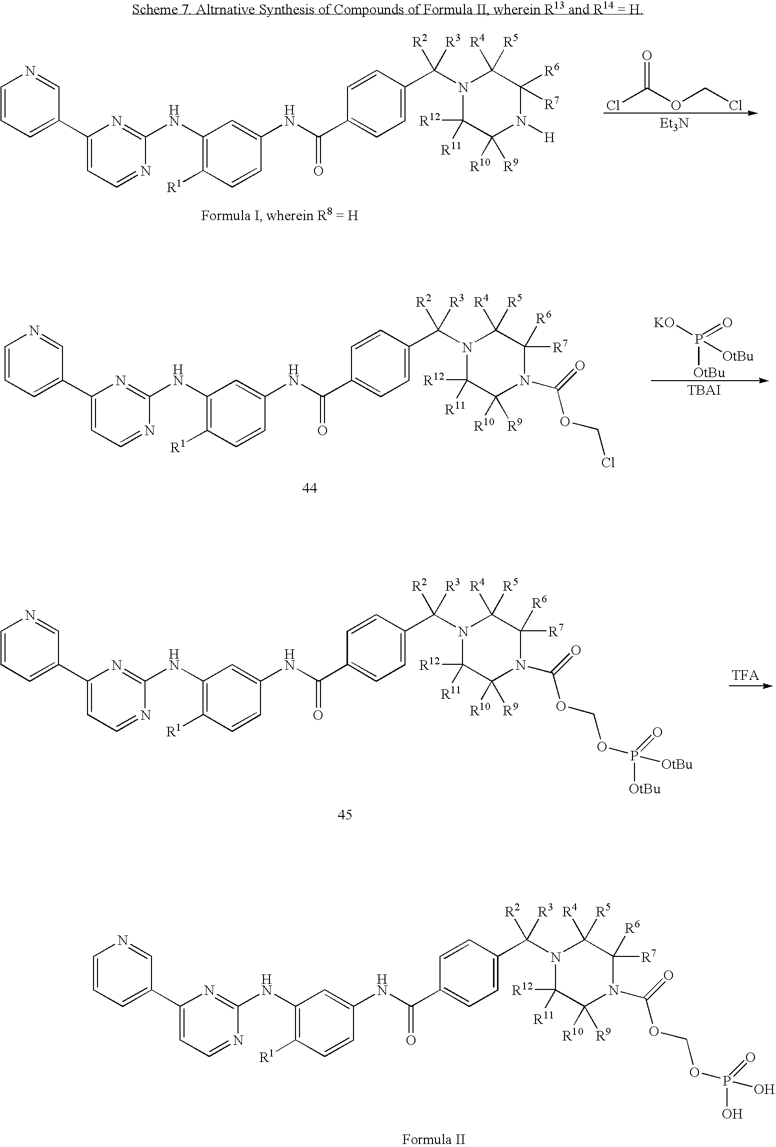

Scheme 7 depicts an alternate method for producing compounds of Formula II. A compound of Formula I wherein R⁸ is hydrogen may be treated with chloromethyl chloroformate to provide compound 44. Treatment with di-tert-butylphosphate potassium salt and tetrabutylammonium iodide (TBAI) provides compound 45. Removal of the t-butyl groups via treatment with trifluoroacetic acid provides compounds of Formula II wherein X is —PO₃H₂ and R¹³ and R¹⁴ are hydrogen. The disodium or calcium salt forms may be accessed via treatment with sodium hydroxide or with calcium hydroxide or calcium acetate or calcium chloride.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and II and their synthetic precursors, including those within routes not explicitly shown in schemes herein, are within the means of chemists of ordinary skill in the art. The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof

Combinations of substituents and variables envisioned by this disclosure are only those that result in the formation of stable compounds.

### Compositions

The disclosure also provides pyrogen-free compositions comprising an effective amount of a compound of Formula I or II, or a pharmaceutically acceptable salt of said compound; and an acceptable carrier. Preferably, a composition of this disclosure is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present disclosure in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this disclosure optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the disclosure include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present disclosure suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this disclosure may be administered in the form of suppositories for rectal administration. These compositions can be prepared by mixing a compound of this disclosure with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this disclosure is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this disclosure include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this disclosure may also be topically applied to the lower intestinal tract by rectal suppository formulation or in a suitable enema formulation. Topically-transdermal patches and iontophoretic administration are also included in this disclosure.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this disclosure may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the disclosure provides a method of coating an implantable medical device comprising the step of contacting said device with the coating composition described above. It will be obvious to those skilled in the art that the coating of the device will occur prior to implantation into a mammal.

According to another embodiment, the disclosure provides a method of impregnating an implantable drug release device comprising the step of contacting said drug release device with a compound or composition of this disclosure. Implantable drug release devices include, but are not limited to, biodegradable polymer capsules or bullets, non-degradable, diffusible polymer capsules and biodegradable polymer wafers.

According to another embodiment, the disclosure provides an implantable medical device coated with a compound or a composition comprising a compound of this disclosure, such that said compound is therapeutically active.

According to another embodiment, the disclosure provides an implantable drug release device impregnated with or containing a compound or a composition comprising a compound of this disclosure, such that said compound is released from said device and is therapeutically active.

Where an organ or tissue is accessible because of removal from the patient, such organ or tissue may be bathed in a medium containing a composition of this disclosure, a composition of this disclosure may be painted onto the organ, or a composition of this disclosure may be applied in any other convenient way.

In another embodiment, a composition of this disclosure further comprises a second therapeutic agent. The second therapeutic agent may be selected from any compound or therapeutic agent known to have or that demonstrates advantageous properties when administered with a compound having the same mechanism of action as imatinib. Such agents include those indicated as being useful in combination with imatinib, including but not limited to, those described in WO 2001047507, WO 2002092091, WO 2002067941, WO 2003013540, WO 2003037322, WO 2003072137, WO 2003084543, WO 2004004644, WO 2004024160, WO 2004026234, WO 2005020972, WO 2005027971, WO 2005049021, WO 2005058320, WO 2005094824, WO 2005072826, WO 2006012958, WO 2006041976, WO 2006065780, WO 2006035204, WO 2006035203, WO 2006052810, WO 2006132930, WO 2007014335, WO 2007051862, and WO 2007052849.

Preferably, the second therapeutic agent is an agent useful in the treatment or prevention of a disease or condition selected from chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

In one embodiment, the second therapeutic agent is selected from hydroxyurea, ranibizumab, homoharringtonine, asparaginase, cyclophosphamide, cytarabine, daunorubicin hydrochloride, etoposide, filgrastim, idarubicin, mercaptopurine, methotrexate, methylprednisolone, mitoxantrone hydrochloride, prednisone, vincristine, TALL-104 cells, cladribine, temsirolimus, alemtuzumab, IFN-alpha, busulfan, fludarabine, clofarabine, xeloda, pioglitazone, etoricoxib, dexamethason, treosulfan, docetaxel, sunitinib, vinorelbine, cisplatin, pemetrexed, temozolomide, vatalanib, everolimus, taxotere, gemcitabine, and capecitabine.

In another embodiment, the disclosure provides separate dosage forms of a compound of this disclosure and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the disclosure, the compound of the present disclosure is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this disclosure can range from about 2 to 8000 mg per treatment. In a more specific embodiment the range is from about 20 to 4000 mg, or from about 40 to 1600 mg, or most specifically from about 200 to 800 mg per treatment. Treatment typically is administered once or twice daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for imatinib.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this disclosure. When this occurs, it will allow the effective dosage of the second therapeutic agent and/or the compound of this disclosure to be reduced from that required in a monotherapy. This has the advantage of minimizing toxic side effects of either the second therapeutic agent of a compound of this disclosure, synergistic improvements in efficacy, improved ease of administration or use and/or reduced overall expense of compound preparation or formulation.

### Methods of Treatment

In another embodiment, the disclosure provides a method of inhibiting protein-tyrosine kinase in a cell, comprising contacting a cell with one or more compounds of Formula I or II herein or pharmaceutically acceptable salts thereof.

According to another embodiment, the disclosure provides a method of treating a patient suffering from, or susceptible to, a disease that is beneficially treated by imatinib comprising the step of administering to said patient an effective amount of a compound of Formula I or II or pharmaceutically acceptable salts thereof or a composition of this disclosure. Such diseases are well known in the art and are disclosed in, but not limited to the following patents and published applications: U.S. Pat. No. 5,521,184, WO 2003068265, WO 2003080061, WO 2003094904, WO 2004096225, WO 2004105763, WO 2005007687, WO 2005020972, WO 2005049021, WO 2005070406, WO 2005072826, WO 2005102318, WO 2005102325, WO 2005102326, WO 2005102455, WO 2005102346, WO 2005112920, WO 2005115304, WO 2005117885, WO 2006042362, WO 2006124544, WO 2007003411, WO 2007014943, and WO 2007051862. Such diseases include, but are not limited to, skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

In one particular embodiment, the method of this disclosure is used to treat a patient suffering from or susceptible to a disease or condition selected from chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

In another particular embodiment, the method of this disclosure is used to treat a patient suffering from or susceptible to a disease or condition selected from chronic myeloid leukemia, gastrointestinal stromal cancer (GIST), fibrosarcoma, acute lymphocytic leukemia, hypereosinophilic syndrome, myeloproliferative diseases, and systemic mastocytosis.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to said patient one or more second therapeutic agents. The choice of second therapeutic agent may be made from any second therapeutic agent known to be useful for co-administration with imatinib. The choice of second therapeutic agent is also dependent upon the particular disease or condition to be treated. Examples of second therapeutic agents that may be employed in the methods of this disclosure are those set forth above for use in combination compositions comprising a compound of this disclosure and a second therapeutic agent.

In particular, the combination therapies of this disclosure include co-administering an effective amount of a compound of Formula I or II or pharmaceutically acceptable salts thereof and of a second therapeutic agent for treatment of the following conditions: leukemia (homoharringtonine, asparaginase, cyclophosphamide, cytarabine, daunorubicin hydrochloride, etoposide, filgrastim, idarubicin, mercaptopurine, methotrexate, methylprednisolone, mitoxantrone hydrochloride, prednisone, vincristine, TALL-104 cells, cladribine, temsirolimus, alemtuzumab, IFNalpha, busulfan, fludarabine, clofarabine); glioblastoma multiforme (hydroxyurea); choroidal neovascularization (ranibizumab); colorectal cancer (xeloda); prostate cancer (pioglitazone, etoricoxib, dexamethason, treosulfan, docetaxel); gastrointestinal stromal tumor (sunitinib); breast cancer (vinorelbine, docetaxel); mesothelioma (cisplatin, pemetrexed); brain and central nervous system tumors or glioma (temozolomide, vatalanib, hydroxyurea); renal cancer (everolimus); head and neck cancer (docetaxel); lung cancer (taxotere); solid tumors (gemcitabine, capecitabine); and fibrosis (organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids)) (pirfenidone, pentoxyfylline, and antibodies against TGF-beta, CTGF, and alpha-v-beta-6).

The term “co-administered” as used herein means that the second therapeutic agent may be administered together with a compound of this disclosure as part of a single dosage form (such as a composition of this disclosure comprising a compound of the disclosure and an second therapeutic agent as described above) or as separate, multiple dosage forms. Alternatively, the additional agent may be administered prior to, consecutively with, or following the administration of a compound of this disclosure. In such combination therapy treatment, both the compounds of this disclosure and the second therapeutic agent(s) are administered by conventional methods. The administration of a composition of this disclosure, comprising both a compound of the disclosure and a second therapeutic agent, to a patient does not preclude the separate administration of that same therapeutic agent, any other second therapeutic agent or any compound of this disclosure to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the disclosure, where a second therapeutic agent is administered to a subject, the effective amount of the compound of this disclosure is less than its effective amount would be where the second therapeutic agent is not administered. In another embodiment, the effective amount of the second therapeutic agent is less than its effective amount would be where the compound of this disclosure is not administered. In this way, undesired side effects associated with high doses of either agent may be minimized. Other potential advantages (including without limitation improved dosing regimens and/or reduced drug cost) will be apparent to those of skill in the art.

In yet another aspect, the disclosure provides the use of a compound of Formula I or II alone or together with one or more of the above-described second therapeutic agents in the manufacture of a medicament, either as a single composition or as separate dosage forms, for treatment or prevention in a patient of a disease, disorder or symptom set forth above. Another aspect of the disclosure is a compound of Formula I or II for use in the treatment or prevention in a patient of a disease, disorder or symptom thereof delineated herein.

### Diagnostic Methods and Kits

The compounds and compositions of this disclosure are also useful as reagents in methods for determining the concentration of imatinib in solution or biological sample such as plasma, examining the metabolism of imatinib and other analytical studies.

According to one embodiment, the disclosure provides a method of determining the concentration, in a solution or a biological sample, of imatinib, comprising the steps of:

a) adding a known concentration of a compound of Formula Ito the solution of biological sample;

b) subjecting the solution or biological sample to a measuring device that distinguishes imatinib from a compound of Formula I;

c) calibrating the measuring device to correlate the detected quantity of the compound of Formula I with the known concentration of the compound of Formula I added to the biological sample or solution; and

d) measuring the quantity of imatinib in the biological sample with said calibrated measuring device; and

e) determining the concentration of imatinib in the solution of sample using the correlation between detected quantity and concentration obtained for a compound of Formula I.

Measuring devices that can distinguish imatinib from the corresponding compound of Formula I include any measuring device that can distinguish between two compounds that differ from one another in isotopic abundance. Exemplary measuring devices include a mass spectrometer, NMR spectrometer, or IR spectrometer.

In another embodiment, a method for determining the amount of imatinib in a solution or a biological sample is provided, comprising:

a) adding a known amount of a compound of Formula I to the solution or biological sample;

b) detecting at least one signal for a compound of Formula I and at least one signal for imatinib in a measuring device that is capable of distinguishing the two compounds;

c) correlating the at least one signal detected for a compound of Formula I with the known amount of the compound of Formula I added to the solution or the biological sample; and

d) determining the amount of imatinib in the solution or biological sample using the correlation between the at least one signal detected of the compound of Formula I and the amount added to the solution or biological sample of a compound of Formula I.

In another embodiment, the disclosure provides a method of evaluating the metabolic stability of a compound of Formula I comprising the steps of contacting the compound of Formula I with a metabolizing enzyme source for a period of time and comparing the amount of the compound of Formula I with the metabolic products of the compound of Formula I after the period of time.

In a related embodiment, the disclosure provides a method of evaluating the metabolic stability of a compound of Formula I in a patient following administration of the compound of Formula I. This method comprises the steps of obtaining a serum, blood, tissue, urine or feces sample from the patient at a period of time following the administration of the compound of Formula Ito the subject; and comparing the amount of the compound of Formula I with the metabolic products of the compound of Formula I in the serum, blood, tissue, urine or feces sample.

The present disclosure also provides kits for use to treat chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids). These kits comprise (a) a pharmaceutical composition comprising a compound of Formula I or II or a salt thereof, wherein said pharmaceutical composition is in a container; and (b) instructions describing a method of using the pharmaceutical composition to treat a disease selected from chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this disclosure may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this disclosure may comprise in a separate vessel of container a pharmaceutical composition comprising a second therapeutic agent, such as one of those listed above for use for co-administration with a compound of this disclosure.

### EXAMPLES

### Example 1

### Synthesis of 4-(Chloromethyl-d₂)benzoyl chloride (15-d₂)

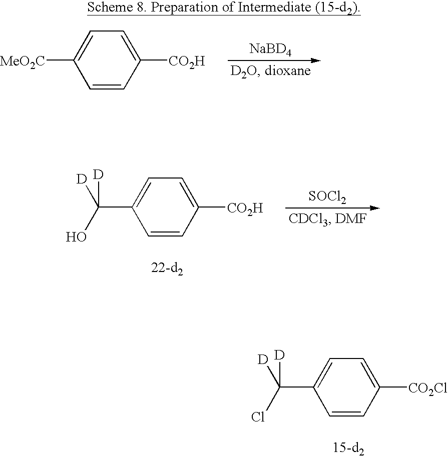

Step 1. 4-(Hydroxymethyl-d₂)benzoic acid (22-d₂). A round-bottomed flask was charged with 4-(methoxycarbonyl)benzoic acid (5.0 g, 27.8 mmol), 1,4-dioxane (69.5 mL), and D₂O (69.5 mL). The mixture was cooled to 0° C. and then NaBD₄ (8.15 g, 195 mmol) was added in eight 1.019 g batches (note: vigorous bubbling resulted from addition). The mixture was heated at 50° C. for 6 hours. The mixture was cooled to room temperature and then to 0° C. The reaction was quenched by the addition of 1N DCl in D₂O (approximately 300 mL). Vigorous bubbling was observed. The mixture was diluted with EtOAc, stirred 5 minutes at room temperature, and then the organic layer was washed with 1N DCl (2×). The combined aqueous solutions were washed with EtOAc (3×). The combined organic solutions were dried (Na₂SO₄), filtered through Celite and concentrated in vacuo. Purification via automated flash column chromatography (80 g SiO₂, 0 to 100% EtOAc in heptanes) afforded 22-d₂ (2.25 g, 52%). MS (M−H): 153.1.

Step 2. 4-(Chloromethyl-d₂)benzoyl chloride (15-d₂). A round-bottomed flask was charged with 22-d₂ (2.15 g, 14.0 mmol), CDCl₃ (20.8 mL), SOCl₂ (2.25 mL, 30.8 mmol) and DMF (0.207 mL). The mixture was heated at reflux for 3 hours. The mixture was then cooled to room temperature and concentrated nearly to dryness in vacuo. The residue was diluted with toluene and concentrated in vacuo again. This toluene azeotroping procedure was repeated, and the resulting crude material was placed under high vacuum to afford 15-d₂ (2.67 g, >99%). MS (M−C+O): 171.1.

### Example 2

### Synthesis of N-(4-Methyl-3-(4-(pyridin-3-yl)pyrimidin-2-ylamino)phenyl)-4-((piperazin-1-yl-d₈)methyl-d₂)benzamide (111)

Compound 111 was prepared as generally outlined in Scheme 1 starting from intermediate 14-d₀.

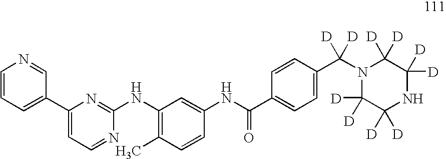

Step 1. 4-(Chloromethyl-d₂)-N-(4-methyl-3-(4-(pyridin-3-yl)pyrimidin-2-ylamino)phenyl)benzamide (16a). 6-Methyl-N¹-(4-(pyridin-3-yl)pyrimidin-2-yl)benzene-1,3-diamine (14-d₀) (0.068 g, 0.245 mmol, prepared according to the procedure described in Kompella, A. et al., WO 2004108699) was dissolved in CDCl₃ (0.425 mL) and Et₃N (0.066 mL) was added. A solution of 3 (56 mg, 0.292 mmol) in CDCl₃ (0.372 mL) was added via cannula. The reaction was stirred overnight, filtered and the filter cake rinsed with H₂O, then EtOAc. The filter cake was air-dried for 10 minutes then transferred to a vial and dried in a vacuum oven (50° C.) for several hours to afford 16a (64 mg, 60%). MS (M+H): 432.1.

Step 2. N-(4-Methyl-3-(4-(pyridin-3-yl)pyrimidin-2-ylamino)phenyl)-4-((piperazin-1-yl-d₈)methyl-d₂)benzamide (111). To a solution of 16a (0.387 g, 0.896 mmol) in CH₂Cl₂ (2.85 mL) was added a solution of piperazine-d8 (0.413 g, CDN Isotopes, 98 atom % D) in MeCN (42.3 mL) via cannula. The reaction was stirred at reflux overnight, then cooled to room temperature and filtered. The solid was partitioned between 1N NaOD in D₂O and 80:20 CHCl₃/MeOH. The organic layer was washed twice with 1N NaOD in D₂O. The combined aqueous solutions were back-extracted twice with 80:20 CHCl₃/MeOH. The combined organic solutions were filtered, concentrated in vacuo and purified via automated flash column chromatography (24 g silica gel, 20 to 100% MeOH in CH₂Cl₂). The fractions containing the desired product were concentrated in vacuo, placed under high vacuum for 5 minutes, then dissolved in acetone (12.7 mL), and filtered. The filtrate was treated with methanesulfonic acid (0.0519 mL). The resulting precipitate was filtered to afford a pale yellow solid. The solid was suspended in benzene, the suspension frozen at −78° C. and the benzene was removed by lyophilization overnight. This procedure afforded the bis-methanesulfonic acid salt of Compound 111 (203 mg, 33%) as a pale yellow solid. ¹H NMR (DMSO-d₆, 400 MHz): δ 10.27 (s, 1H), 9.32 (s, 1H), 9.07 (s, 1H), 8.77-8.71 (m, 2H), 8.64-8.57 (m, 1H), 8.56-8.52 (m, 1H), 8.10-8.08 (m, 1H), 8.07-7.99 (m, 1H), 7.67-7.56 (m, 2H), 7.50-7.45 (m, 2H), 7.22 (d, J=8.3, 1H), 2.32 (s, 3H), 2.31 (s, 3H), 2.23 (s, 3H). MS: m/z=245.7. Observed value corresponds to (M+2H)/z where z is 2.

### Example 3

### Synthesis of N-(4-(Methyl-d₃)-3-(4-(pyridin-3-yl)pyrimidin-2-ylamino)phenyl)-4-((piperazin-1-yl-d₈)methyl-d₂)benzamide (102)

Compound 102 was prepared as generally outlined in Scheme 1 starting from intermediate 13-d₃. Intermediate 13-d₃ was generated from commercially available 13-d₀ as described in step 1 below.

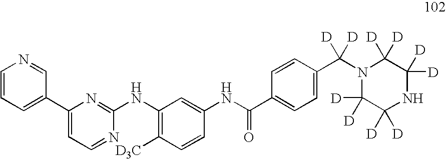

Step 1. N-(2-(Methyl-d₃)-5-nitrophenyl)-4-(pyridin-3-yl)pyrimidin-2-amine (13-d₃). A round-bottomed flask was charged with commercially available N-(2-methyl-5-nitrophenyl)-4-(pyridin-3-yl)pyrimidin-2-amine (13-d₀) (2.0 g, 6.51 mmol), THF (21.7 mL), MeOD (21.7 mL, Sigma-Aldrich, 99.9% D), D₂O (21.7 mL) and K₂CO₃ (9.04 g). The mixture was heated at reflux for approximately 18 hours. The mixture was concentrated in vacuo nearly to dryness, charged with THF (21.7 mL), MeOD (21.7 mL, Sigma-Aldrich, 99.9% D), D₂O (21.7 mL) and K₂CO₃ (9.04 g), and heated at reflux for another 18 hours. The reaction was then cooled to room temperature and partitioned between CH₂Cl₂ and D₂O. The aqueous layer was washed with CH₂Cl₂ (3×) and then the combined organic solutions were washed with D₂O (1×). The combined organic solutions were dried (Na₂SO₄), filtered and concentrated in vacuo. Purification via automated flash column chromatography (40 g SiO₂, 30 to 100% EtOAc in heptanes) afforded 13-d₃ (895 mg, 44%). MS (M+H): 311.1.

Step 2. 6-(Methyl-d₃-N¹-(4-(pyridin-3-yl)pyrimidin-2-1)benzene-1,3-diamine 14-d₃. A round-bottomed flask was charged with 35 wt. % DCl in D₂O (14.1 mL, Sigma-Aldrich, 99% D), and 5 nCl₂.2H₂O. The mixture was stirred for 10 minutes, cooled to 0° C., and 13-d₃ (1.34 g, 4.32 mmol) was added. The mixture was heated at 30° C. for approximately 18 hours. The mixture was transferred to a reparatory funnel with D₂O (374 mL), 40% NaOD in D₂O (16.8 mL) was added, and the mixture was extracted with CHCl₃ (3×). The combined organic solutions were dried (Na₂SO₄), filtered and concentrated in vacuo. Purification via automated flash column chromatography (40 g SiO₂, 0 to 10% MeOH in CH₂Cl₂) afforded 14-d₃ (1.05 g, 87%). MS (M+H): 281.2.

Step 3. 4-(Chloromethyl-d₂)-N-(4-(methyl-d₃)-3-(4-(pyridin-3-yl)pyrimidin-2-ylamino)phenyl)benzamide (16b). 14-d₃ (0.585 mg, 2.09 mmol) was dissolved in CDCl₃ (3.62 mL) and Et₃N (0.562 mL) was added. A solution of 15-d₂ (477 mg) in CDCl₃ (3.17 mL) was added via cannula. The reaction was stirred overnight, filtered and the filter cake rinsed with D₂O and then EtOAc. The filter cake was air-dried for 10 minutes then transferred to a vial and dried in a vacuum oven (50° C.) for several hours to afford 16b (592 mg, 65%) contaminated with ˜10% triethylammonium chloride. MS (M+H): 435.1.

Step 4. N-(4-(Methyl-d₃)-3-(4-(pyridin-3-yl)pyrimidin-2-ylamino)phenyl)-4-((piperazin-1-yl-dₛ)methyl-d₂)benzamide (102). To a solution of 16b (0.592 g, 1.36 mmol) in CH₂Cl₂ (4.33 mL) was added a solution of piperazine-d8 (0.628 g, CDN Isotopes, 98 atom % D) in MeCN (64.3 mL) via cannula. The reaction was stirred at reflux overnight, then cooled to room temperature and filtered. The solid was partitioned between 1N NaOD in D₂O and 80:20 CHCl₃/MeOH. The organic layer was washed twice with 1N NaOD in D₂O. The combined aqueous solutions were back-extracted twice with 80:20 CHCl₃/MeOH. The combined organic solutions were filtered, concentrated in vacuo and purified via automated flash column chromatography (40 g silica gel, 30 to 100% MeOH in CH₂Cl₂, doped with 1% conc. NH₄OH). The fractions containing the desired product were concentrated in vacuo, placed under high vacuum for 5 minutes, then dissolved in CH₂Cl₂ (˜30 mL), and filtered. The filtrate was treated with methanesulfonic acid (0.099 mL). The resulting precipitate was filtered to afford a pale yellow solid. The solid was suspended in benzene, the suspension frozen at −78° C. and the benzene was removed by lyophilization overnight to afford the bis-methanesulfonic acid salt of Compound 102 (403 mg, 43%) as a pale yellow solid. ¹H NMR (DMSO-d6, 400 MHz): δ 10.29 (s, 1H), 9.35 (s, 1H), 9.10 (s, 1H), 8.78 (d, J=4.0, 2H), 8.67 (d, J=6.6, 1H), 8.56 (d, J=4.0, 1H), 8.10 (s, 1H), 8.08-7.99 (m, 1H), 7.74-7.58 (m, 2H), 7.52-7.44 (m, 2H), 7.22 (d, J=8.1, 1H), 2.34 (s, 6H). MS: m/z=247.2. Observed value corresponds to (M+2H)/z where z is 2.

### Example 4

### Synthesis of N-(4-(Methyl-d₃)-3-(4-(pyridin-3-yl)pyrimidin-2-ylamino)phenyl)-4-(piperazin-1-yl(methyl-d₂))benzamide (108)

Compound 108 was prepared as generally outlined in Scheme 1 starting from intermediate 16b.

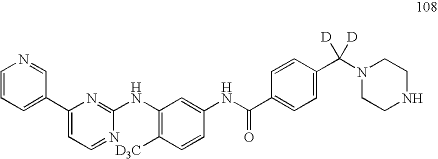

N-(4-(Methyl-d₃)-3-(4-(pyridin-3-yl)pyrimidin-2-ylamino)phenyl)-4-(piperazin-1-yl(methyl-d₂))benzamide (108). To a mixture of MeCN (66 mL) and CH₂Cl₂ (4 mL) was added piperazine (0.543 g, 6.30 mmol) then 16b (0.550 g, 1.26 mmol, see Example 3, steps 1-3 for preparation). The reaction was stirred at reflux overnight, then cooled to room temperature and filtered. The solid was partitioned between 1N NaOD in D₂O and 80:20 CHCl₃/MeOH. The organic layer was washed twice with 1N NaOD in D₂O. The combined aqueous solutions were back-extracted twice with 80:20 CHCl₃/MeOH. The combined organic solutions were filtered, concentrated in vacuo and purified via automated flash column chromatography (40 g silica gel, 30 to 100% MeOH in CH₂Cl₂). The fractions containing the desired product were concentrated in vacuo, placed under high vacuum for 5 minutes, then dissolved in CH₂Cl₂ (30 mL), and filtered. The filtrate was treated with methanesulfonic acid (0.093 mL). The resulting precipitate was filtered to afford a pale yellow solid, which was determined to be the mono-methanesulfonic acid salt of Compound 108 by ¹H NMR analysis. This salt was suspended in CH₂Cl₂ (30 mL) and treated with methanesulfonic acid (0.047 mL). The resulting precipitate was filtered and suspended in benzene. The suspension was frozen at −78° C. and the benzene was removed by lyophilization overnight. This procedure afforded the bis-methanesulfonic acid salt of Compound 108 (366 mg, 60%) as a pale yellow solid. ¹H NMR (DMSO-d6, 400 MHz): δ 10.29 (s, 1H), 9.36 (s, 1H), 9.11 (s, 1H), 8.79 (d, J=4.5, 2H), 8.71 (d, J=8.6, 1H), 8.56 (d, J=5.6, 1H), 8.10 (s, 1H), 8.05 (d, J=7.6, 2H), 7.77-7.70 (m, 1H), 7.69-7.60 (m, 2H), 7.53-7.44 (m, 2H), 7.23 (d, J=8.6, 1H), 3.74-2.79 (br s, 8H), 2.34 (s, 6H). MS: m/z=243.3. Observed value corresponds to (M+2H)/z where z is 2.

### Example 5

### Synthesis of N-(4-(Methyl-d₃)-3-(4-(pyridin-3-yl)pyrimidin-2-ylamino)phenyl)-4-(piperazin-1-ylmethyl)benzamide (120)

Compound 120 was prepared as generally outlined in Scheme 1 starting from intermediate 14-d₃.

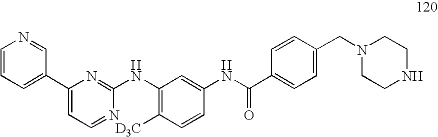

Step 1. 4-(chloromethyl)-N-(4-methyl-3-(4-(pyridin-3-yl)pyrimidin-2-ylamino)phenyl)benzamide (16c). 14-d₃ (0.551 mg, 1.97 mmol, see Example 3, steps 1-2 for preparation) was dissolved in CDCl₃ (3.41 mL) and Et₃N (0.529 mL) was added. A solution of 15-d₀ (446 mg, commercially available) in CDCl₃ (2.98 mL) was added via cannula. The reaction was stirred overnight, filtered and the filter cake rinsed with D₂O and then EtOAc. The filter cake was air-dried for 10 minutes then transferred to a vial and dried in a vacuum oven (50° C.) for several hours to afford 16c (559 mg, 66%) contaminated with ˜10% triethylammonium chloride. MS (M+H): 433.1.

Step 2. N-(4-(Methyl-d₃)-3-(4-(pyridin-3-yl)pyrimidin-2-ylamino)phenyl)-4-(piperazin-1-ylmethyl)benzamide (120). To a mixture of MeCN (67 mL) and CH₂Cl₂ (4 mL) was added piperazine (0.548 g) then 16c (0.550 g). The reaction was stirred at reflux overnight, then cooled to room temperature and filtered. The solid was partitioned between 1N NaOD in D₂O and 80:20 CHCl₃/MeOH. The organic layer was washed twice with 1N NaOD in D₂O. The combined aqueous solutions were back-extracted twice with 80:20 CHCl₃/MeOH. The combined organic solutions were filtered, concentrated in vacuo and purified via automated flash column chromatography (40 g silica gel, 0 to 100% MeOH in CH₂Cl₂). The fractions containing the desired product were concentrated in vacuo, placed under high vacuum for 5 minutes, then dissolved in acetone (43 mL), and filtered. The filtrate was treated with methanesulfonic acid (0.098 mL). The resulting precipitate was filtered to afford a pale yellow solid. The solid was suspended in benzene, the suspension frozen at −78° C. and the benzene was removed by lyophilization overnight. Further drying in a vacuum oven (50° C.) afforded the bis-methanesulfonic acid salt of Compound 120 (476 mg, 56%) as a pale yellow solid. ¹H NMR (DMSO-d6, 400 MHz): δ 10.28 (s, 1H), 9.35 (s, 1H), 9.09 (s, 1H), 8.91-8.80 (br s, 1H), 8.78 (d, J=4.3, 2H), 8.67 (d, J=8.6, 1H), 8.56 (d, J=5.4, 1H), 8.10 (s, 1H), 8.05 (d, J=7.5, 2H), 7.73-7.67 (m, 1H), 7.69-7.60 (m, 2H), 7.52-7.44 (m, 2H), 7.22 (d, J=9.2, 1H), 4.54-3.97 (br s, 2H), 3.54-2.84 (br s, 8H), 2.36 (s, 6H). MS: m/z=242.2. Observed value corresponds to (M+2H)/z where z is 2.

### Evaluation of Metabolic Stability

Certain in vitro liver metabolism studies have been described previously in the following references, each of which is incorporated herein in their entirety: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al., Pharmacol Ther, 1997, 73:147; and Lave, T, et al., Pharm Res, 1997, 14:152.

Microsomal Assay: The metabolic stability of compounds of Formula I or II is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Human liver microsomes are obtained from a commercial source (e.g., XenoTech, LLC (Lenexa, Kans.)). The incubation mixtures are prepared as follows:

### Reaction Mixture Composition

| | | |
|---|---|---|
| Liver Microsomes | 0.5-2.0 | mg/mL |
| NADPH | 1 | mM |
| Potassium Phosphate, pH 7.4 | 100 | mM |
| Magnesium Chloride | 10 | mM |
| Test Compound | 0.1-1 | μM. |

Incubation of Test Compounds with Liver Microsomes: The reaction mixture, minus cofactors, is prepared. An aliquot of the reaction mixture (without cofactors) is incubated in a shaking water bath at 37° C. for 3 minutes. Another aliquot of the reaction mixture is prepared as the negative control. The test compound is added into both the reaction mixture and the negative control at a final concentration of 0.1-1 μM. An aliquot of the reaction mixture is prepared as a blank control, by the addition of plain organic solvent (no test compound). The reaction is initiated by the addition of cofactors (not added to negative controls), and then incubated in a shaking water bath at 37° C. Aliquots (200 μL) are withdrawn in triplicate at multiple time points (e.g., 0, 15, 30, 60, and 120 minutes) and combined with 800 μL of ice-cold 50/50 acetonitrile/dH₂O to terminate the reaction. The positive controls, testosterone and propranolol, as well as Compound 1, are each run simultaneously with the test compounds in separate reactions.

All samples are analyzed using LC-MS (or MS/MS). An LC-MRM-MS/MS method is used for metabolic stability. Also, Q1 full scan LC-MS methods are performed on the blank matrix and the test compound incubation samples. The Q1 scans serve as survey scans to identify any sample unique peaks that might represent the possible metabolites. The masses of these potential metabolites can be determined from the Q1 scans.

SUPERSOMES™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula I or II in 100 mM potassium phosphate buffer (pH 7.4) is incubated at 37° C. in triplicate. Positive controls contain 1 μM of imatinib instead of a compound of Formula I or II. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 504 of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

Without further description, it is believed that one of ordinary skill in the art can, using the preceding description and the illustrative examples, make and utilize the compounds of the present disclosure and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the disclosure. All the patents, journal articles and other documents discussed or cited above are herein incorporated by reference.

1. A compound of Formula I: 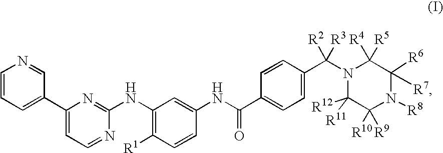
or a pharmaceutically acceptable salt thereof, wherein:
R¹ is selected from CH₃, CH₂D, CHD₂, and CD₃;
each of R², R³, R⁴, R⁵, R⁶, R⁷, R⁹, R¹⁰, R¹¹ and R¹² is independently selected from H and D;
R⁸ is hydrogen; and
at least one R comprises a deuterium atom,
wherein:
when R² and R³ are simultaneously hydrogen, and R⁴, R⁵, R⁶, R⁷, R⁹, R¹⁰, R¹¹ and R¹² are simultaneously deuterium, then R¹ comprises a deuterium atom.

2. The compound of claim 1, wherein R⁸ is H.

3. A compound of Formula II: 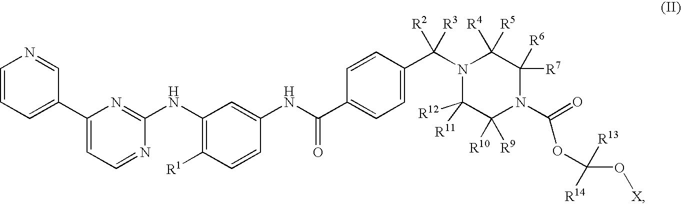
or a pharmaceutically acceptable salt thereof, wherein:
R¹ is selected from CH₃, CH₂D, CHD₂, and CD₃;
each of R², R³, R⁴, R⁵, R⁶, R⁷, R⁹, R¹⁰, R¹¹ and R¹² is independently selected from hydrogen and deuterium;
each of R¹³ and R¹⁴ is independently selected from hydrogen, deuterium, C₁-C₆-alkyl, C₂-C₆ alkenyl, C₂-C₆ alkynyl, C₃-C₆ cycloalkyl, C₅-C₆ cycloalkenyl, and C₄-C₈ (cycloalkyl)alkyl; or
R¹³ and R¹⁴ are taken together with the carbon atom to which they are bound to form a 3-to 7-membered carbocyclic ring; and
X is selected from —PO₃H₂ (including a pharmaceutically acceptable salt of —PO₃H₂) and -A-R¹⁵, wherein A is an α-amino acid residue and R¹⁵ is selected from hydrogen, —CH₃, —C(O)CH₃, and an α-amino acid.

4. The compound of claim 3, wherein each of R¹³ and R¹⁴ is independently selected from hydrogen and —CH₃.

5. The compound of claim 3, wherein X is selected from a pharmaceutically acceptable salt of —PO₃H₂ or -A-R¹⁵, wherein A is a naturally occurring α-amino acid and R¹⁵ is hydrogen.

6. The compound of claim 5, wherein X is a pharmaceutically acceptable salt of —PO₃H₂.

7. The compound of claim 1 or 3, wherein each R group attached to a common carbon atom is the same.

8. The compound of claim 7, wherein R⁴, R⁵, R⁶, R⁷, R⁹, R¹⁰, R¹¹ and R¹² are the same.

9. The compound of claim 1 or 3, wherein R¹ is selected from CH₃ and CD₃.

10. The compound of claim 1, wherein R² and R³ are the same; each of R⁴, R⁵, R⁶, R⁷, R⁹, R¹⁰, R¹¹ and R¹² are the same, and the compound is selected from any one of the compounds set forth in the table:

| Compound | R¹ | R²=R³ | R⁴=R⁵=R⁶=R⁷=R⁹=R¹⁰=R¹¹=R¹² | R⁸ |
|---|---|---|---|---|
| 102 | CD₃ | D | D | H |
| 105 | CD₃ | H | D | H |
| 108 | CD₃ | D | H | H |
| 111 | CH₃ | D | D | H |
| 114 | CH₃ | H | D | H |

or a pharmaceutically acceptable salt of any of the foregoing.

11. The compound of claim 1, wherein the compound is 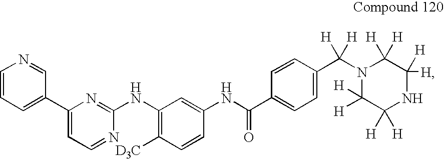
or a pharmaceutically acceptable salt thereof.

12. The compound of claim 3, wherein R² and R³ are the same; each of R⁴, R⁵, R⁶, R⁷, R⁹, R¹⁰, R¹¹ and R¹² is the same; R¹³ and R¹⁴ are hydrogen; where the compound is selected from any one of the compounds set forth in the table:

| | | | R⁴=R⁵=R⁶= | |
|---|---|---|---|---|
| Compound | R¹ | R²=R³ | R⁷=R⁹=R¹⁰=R¹¹=R¹² | X |
| 115 | CD₃ | D | H | PO₃H₂ |
| 116 | CD₃ | H | H | PO₃H₂ |
| 117 | CD₃ | D | D | PO₃H₂ |
| 118 | CH₃ | D | H | PO₃H₂ |
| 119 | CH₃ | H | H | PO₃H₂ |

or a pharmaceutically acceptable salt of any of the foregoing.

13. The compound of claim 1 or 3, wherein any atom not designated as deuterium is present at its natural isotopic abundance.

14. A pyrogen-free composition comprising:
a. a compound of Formula I: 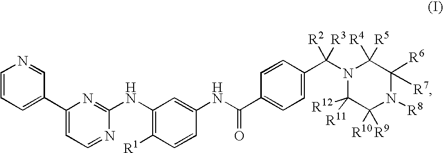
or a pharmaceutically acceptable salt thereof, wherein:
R¹ is selected from CH₃, CH₂D, CHD₂, and CD₃;
each of R², R³, R⁴, R⁵, R⁶, R⁷, R⁹, R¹⁰, R¹¹ and R¹² are independently selected from hydrogen and deuterium;
R⁸ is hydrogen; and
at least one R comprises a deuterium atom; and
b. an acceptable carrier.

15. A pyrogen-free composition comprising:
a. a compound of Formula II: 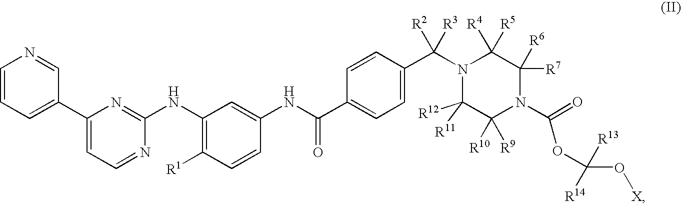
or a pharmaceutically acceptable salt thereof, wherein:
R¹ is selected from CH₃, CH₂D, CHD₂, and CD₃;
each of R², R³, R⁴, R⁵, R⁶, R⁷, R⁹, R¹⁰, R¹¹ and R¹² is independently selected from hydrogen and deuterium;
each of R¹³ and R¹⁴ is independently selected from hydrogen, deuterium, C₁-C₆-alkyl, C₂-C₆ alkenyl, C₂-C₆ alkynyl, C₃-C₆ cycloalkyl, C₅-C₆ cycloalkenyl, and C₄-C₈ (cycloalkyl)alkyl; or
R¹³ and R¹⁴ are taken together with the carbon atom to which they are bound to form an optionally substituted 3- to 7-membered carbocyclic ring; and
X is selected from —PO₃H₂ (including a pharmaceutically acceptable salt of —PO₃H₂) and -A-R¹⁵, wherein A is an α-amino acid residue and R¹⁵ is selected from hydrogen, —CH₃, —C(O)CH₃, and an α-amino acid; and
b. an acceptable carrier.

16. The composition of claim 14 or 15, wherein the composition is formulated for pharmaceutical administration; and wherein the carrier is a pharmaceutically acceptable carrier.

17. The composition of claim 16, additionally comprising a second therapeutic agent, wherein the second therapeutic agent is useful to treat a patient suffering from or susceptible to chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

18. The composition of claim 17, wherein the second therapeutic agent is selected from hydroxyurea, ranibizumab, homoharringtonine, asparaginase, cyclophosphamide, cytarabine, daunorubicin hydrochloride, etoposide, filgrastim, idarubicin, mercaptopurine, methotrexate, methylprednisolone, mitoxantrone hydrochloride, prednisone, vincristine, TALL-104 cells, cladribine, temsirolimus, alemtuzumab, IFN-alpha, busulfan, fludarabine, clofarabine, xeloda, pioglitazone, etoricoxib, dexamethason, treosulfan, docetaxel, sunitinib, vinorelbine, cisplatin, pemetrexed, temozolomide, vatalanib, everolimus, taxotere, gemcitabine, and capecitabine pentoxyfylline, pirfenidone, and antibodies against TGF-beta, CTGF, and alpha-v-beta-6.

19. A method of inhibiting protein-tyrosine kinase activity in a cell, comprising contacting the cell with a compound of claim 1 or claim 3, or pharmaceutically acceptable salts thereof.

20. A method of treating a patient suffering from, or susceptible to, a disease selected from skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids) the method comprising the step of administering to the patient in need thereof a composition of claim 16.

21. The method of claim 20, wherein the patient is suffering from or susceptible to a disease or condition selected from chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

22. The method of claim 21, wherein the patient is suffering from or susceptible to a disease or condition selected from chronic myeloid leukemia, gastrointestinal stromal cancer (GIST), fibrosarcoma, acute lymphocytic leukemia, hypereosinophilic syndrome, myeloproliferative diseases, and systemic mastocytosis.

23. The method of claim 20, comprising the additional step of administering to the patient in need thereof a second therapeutic agent, wherein the second therapeutic agent is useful to treat a condition selected from: chronic myeloid leukemia, gastrointestinal stromal cancer (GIST), fibrosarcoma, acute lymphocytic leukemia, hypereosinophilic syndrome, myeloproliferative diseases, systemic mastocytosis, astrocytoma, glioblastoma multiforme, pulmonary hypertension, cancer, breast cancer, eye cancer, cancer of the head and neck, non-small cell lung cancer, small-cell lung cancer, metastatic cancer, ovarian cancer, testicular cancer, prostate cancer, thyroid cancer, solid tumor cancer, thymic cancer, pancreatic cancer, renal cancer, colorectal cancer, idiopathic pulmonary fibrosis, interstitial lung diseases, Kaposi's sarcoma, melanoma, meningioma, sarcoma, Ewing's sarcoma, neurofibromatosis, oligodendroglioma, chordoma, Polycythemia Vera, allergic rhinitis, scleroderma, rheumatoid arthritis, malignant mesothelioma, skin cancer, renal disorders, malaria, arterial restenosis, disorders of sexual function and reproduction, eye disorders, psoriasis, diabetes type 1 and type 2, cerebral ischemia, hematologic/blood cancer, Multiple Sclerosis, muscular dystrophy, peripheral vascular disease, neurological disorders, fibrodysplasia, viral hepatitis, acne, cardiovascular disorders, chemical or biological agent exposure, cystic fibrosis, atherosclerosis, urinary incontinence, choriocarcinoma, malignant histiocytosis, embryonal carcinoma, endometrial carcinoma, brain microglial tumours, sarcoidosis, Creutzfeldt-Jacob disease, amyotrophic lateral sclerosis, HIV infection, pathogenic infection, and organ fibrosis, including pulmonary fibrosis, idiopathic pulmonary fibrosis neurofibromatosis, Hermansky-Pudlak syndrome, diabetic nephropathy, renal failure, hypertrophic cardiomyopathy (HCM), glomerulosclerosis (FSGS), radiation-induced fibrosis such as osteoradionecrosis, multiple sclerosis, and uterine leiomyomas (fibroids).

24. The method of claim 23, wherein:
a. the patient is suffering from of susceptible to leukemia; and the second therapeutic agent is selected from homoharringtonine, asparaginase, cyclophosphamide, cytarabine, daunorubicin hydrochloride, etoposide, filgrastim, idarubicin, mercaptopurine, methotrexate, methylprednisolone, mitoxantrone hydrochloride, prednisone, vincristine, TALL-104 cells, cladribine, temsirolimus, alemtuzumab, IFNalpha, busulfan, fludarabine, and clofarabine;
b. the patient is suffering from of susceptible to glioblastoma multiforme; and the second therapeutic agent is hydroxyurea;
c. the patient is suffering from of susceptible to choroidal neovascularization; and the second therapeutic agent is ranibizumab;
d. the patient is suffering from of susceptible to colorectal cancer; and the second therapeutic agent is xeloda;
e. the patient is suffering from of susceptible to prostate cancer; and the second therapeutic agent is selected from pioglitazone, etoricoxib, dexamethason, treosulfan, and docetaxel;
f. the patient is suffering from of susceptible to gastrointestinal stromal tumor; and the second therapeutic agent is sunitinib;
g. the patient is suffering from of susceptible to breast cancer; and the second therapeutic agent is selected from vinorelbine and docetaxel;
h. the patient is suffering from of susceptible to mesothelioma; and the second therapeutic agent is selected from cisplatin and pemetrexed;
i. the patient is suffering from of susceptible to brain and central nervous system tumors or glioma; and the second therapeutic agent is selected from temozolomide, vatalanib, and hydroxyurea;
j. the patient is suffering from of susceptible to renal cancer; and the second therapeutic agent is everolimus;
k. the patient is suffering from of susceptible to head and neck cancer; and the second therapeutic agent is docetaxel;
l. the patient is suffering from of susceptible to lung cancer; and the second therapeutic agent is taxotere;
m. the patient is suffering from of susceptible to solid tumors; and the second therapeutic agent is selected from gemcitabine and capecitabine.

25. The composition of claim 15, wherein a first methylene or methenylene group in the alkyl, alkenyl or alkynyl may be bonded to a second methylene or methenylene group in the same alkyl, alkenyl or alkynyl to form an optionally substituted 3- to 7-membered carbocyclic ring.
